(19) United States
(12) Patent Application Publication
(10) Pub. No.: US 2011/0008903 A1
(43) Pub. Date: Jan. 13, 2011

(54) METHODS AND KITS FOR DETERMINING THE OCCURRENCE OF A LIVER DISEASE IN A SUBJECT

(76) Inventors: Valerie Paradis, Paris Cedex (FR); Pierre Bedossa, Paris Cedex (FR); Viviane Trak-Smayra, Paris Cedex (FR)

Correspondence Address: YOUNG & THOMPSON, 209 Madison Street, Suite 500, Alexandria, VA, 22314, US

(21) Appl. No.: 12/920,358

(22) PCT Filed: Mar. 25, 2009
(86) PCT No.: PCT/EP2009/053525

§ 371 Date: Aug. 31, 2010

(30) Foreign Application Priority Data
Mar. 27, 2008 (EP) 08300162.8

(51) Int. Cl. G01N 33/68

(52) U.S. Cl. 436/86

(57) ABSTRACT

The present application relates to methods for determining the occurrence of a liver disease in a subject by particular polypeptides biomarkers, and to kits using such biomarkers.

### FIELD OF THE INVENTION

The present invention concerns the field of proteomics and diagnosis of human liver diseases.

In particular, the present application relates to methods for determining the occurrence of a liver disease in a subject by means of particular polypeptides biomarkers, and to kits using such biomarkers.

### BACKGROUND OF THE INVENTION

Hepatitis is one of the most common and current liver diseases. This pathology implies injury to liver characterised by presence of inflammation and fibrosis in the liver tissue.

It may run a subclinical course when the affected person may not feel ill. The patient becomes unwell and symptomatic when the disease impairs liver functions that include, among other things, screening of harmful substances, regulation of blood composition, and production of bile to help digestion.

A group of viruses known as the hepatitis viruses cause most cases of liver damage worldwide. Hepatitis can also be due to toxics (notably alcohol), other infections or from autoimmune process.

Patients with metabolic syndrome, suffering from central obesity in association with other cardiovascular risk factors, without excessive alcohol consumption, are also likely to develop a particular type of hepatitis known as Non-Alcoholic Fatty Liver Disease (NAFLD).

NAFLD covers a spectrum of liver damage mimicking alcoholic liver disease and potentially leading to cirrhosis. NAFLD is becoming more frequent with the increasing worldwide prevalence of obesity. Obesity-associated insulin resistance results in the accumulation of free fatty acids and triglycerides in hepatocytes. Additional factors, such as excessive production of reactive oxygen species, might induce inflammation and extracellular matrix deposits. Morphologically, patterns of liver damage vary from steatosis to steatohepatitis (NASH), fibrosis, cirrhosis, and even to the development of hepatocellular carcinoma (HCC).

In case of NAFLD, the disease prognosis differs according to the severity of lesions, and the presence of NASH might be a critical factor.

Up to now, no screening test has proven its efficacy at detecting liver damage in obese patients. Its diagnosis relies always on liver biopsy, since liver blood tests may be normal in this context.

Ultrasound and CT scan are efficient at identifying significant steatosis but are not suitable for visualizing liver inflammation and early stages of fibrosis. Even Fibroscan®, a recent ultrasound technique that allows non-invasive quantification of liver fibrosis, is useless in obese patients because the thickness of subcutaneous and abdominal fat reduces the propagation of the waves.

As a result, although biochemical and radiological investigations are suggestive of steatosis, they cannot detect lesions consistent with NASH, a diagnosis that still requires liver biopsy as above-mentioned. But, there is increasing reluctance on the part of both patients and physicians to perform liver biopsy due to its potential risk of complications.

Therefore, several retrospective studies have sought to correlate clinical and biological variables with histopathological patterns in an attempt to search for sensitive and specific non-invasive markers of NASH and advanced fibrosis. Most of those studies pointed out high body mass index (BMI), diabetes mellitus and older age as relevant predictors of deleterious evolution.

However all these diagnostics methods are not totally satisfying. In this setting, a major challenge is to identify patients at risk for deleterious evolution, in order to prevent in particular cirrhosis and its complications.

There is thus a need in the art for non-invasive method and reliable biomarkers, which could allow determining the occurrence of a liver disease in a subject, and also for kits using such biomarkers.

### SUMMARY OF THE INVENTION

The present invention relates to a method for determining the occurrence of a liver disease in a subject comprising the steps of:

a) providing a sample previously collected from the said subject,

b) measuring at least one biomarker in the said sample, wherein the said biomarker is selected from the group of polypeptides consisting of
(i) a positively charged polypeptide at pH 5 having a SELDI-TOF mass-to-charge (m/z) ratio of 7558.4+/−0.3% (according to manufacturer data,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%,

c) determining the occurrence of a liver disease from the biomarker values measured at step b).

At step b) of the determination method above, the positively charged polypeptides (i) and (ii) are preferably measured on a CM10® protein chip array marketed by Ciphergen (Le Raincy, France).

At step b) of the method above, the negatively charged polypeptide (iii) is preferably measured on a Q10® protein chip array marketed by Ciphergen (Le Raincy, France).

In certain embodiments, the liver disease is selected from the group consisting of a chronic liver disease and a non-chronic liver disease.

In other embodiments, the liver disease is selected from steatosis, steatohepatitis, cirrhosis and fibrosis; steatohepatitis consists advantageously in non-alcoholic steatohepatitis (NASH).

In further embodiments, the subject is selected from the group consisting of (i) a subject infected by a hepatitis virus, (ii) an alcoholic subject and (ii) a subject affected with obesity.

According to a preferred characteristic, the sample consists of a serum sample that has been collected from the said subject.

In still further embodiments, the method further comprises a step d) of determining the severity of the liver disease, wherein the severity of the liver disease is determined by measuring the amount of the at least one biomarker measured at step b).

According to these further embodiments, the measure of the amount of the at least one biomarker is advantageously performed by determining the intensity value of the signal that is generated by the said biomarker when the said sample is subjected to a mass spectrum analysis or to a SELDI-TOF analysis.

In yet further embodiments, the method comprises further a step d) of determining the severity of the liver disease by performing the following steps of:
d1) determining the intensity value of the signal that is generated by the said biomarker when the said sample is subjected to a SELDI-TOF analysis,
d2) comparing the signal intensity value obtained at step d1) with at least a reference signal intensity value that is expected to be measured in an individual selected form the group consisting of (i) an individual who is not affected by a liver disease, (ii) an individual who is affected with a liver steatosis and (iii) an individual who is affected with a steatohepatitis, and
d3) determining the severity of the liver disease.

In accordance to these last embodiments, the severity of the liver disease is advantageously determined at step d3) as being the status for which the ratio of the subject to the reference signal intensity values are close to 1, preferably ranging from 0.99 to 1.01.

According to certain other embodiments, the amount of the at least one biomarker is measured by performing an immunoassay.

The invention concerns also a method for monitoring the post-operative status of a patient that has been subjected to a bariatric surgery comprising a step of performing the method above specified, with one or more samples that have been collected from the said patient at one or more instants following the surgical operation.

The invention has also for object the method for the in vivo testing of a substance for treating a liver disease comprising the steps of:
a) administering the said substance to a patient in need of a medical treatment of a liver disease,
b) performing the method as above specified on the said patient, and
c) determining the effect of the said substance on the liver disease of the said patient.

In accordance to the above embodiment, the method may further comprise a step preceding step a) wherein the said method is performed on the said patient before administration of the substance to be tested.

The present invention concerns also kits for determining the occurrence of a liver disease in a subject.

In a first embodiment, the kit comprises means necessary for measuring at least one biomarker in the said sample, wherein the said biomarker is selected from the group of polypeptides consisting of
(i) a positively charged polypeptide at pH 5 having a SELDI-TOF mass-to-charge (m/z) ratio of 7558.4+/−0.3% (according to manufacturer data,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%.

In the detection kit above, the means necessary for measuring the positively charged polypeptides (i) and (ii) preferably consist of a CM10® protein chip array marketed by Ciphergen (Le Raincy, France).

In the detection kit above, the means necessary for measuring the negatively charged polypeptide (iii) preferably consist of a Q10® protein chip array marketed by Ciphergen (Le Raincy, France).

In a second embodiment, the kit comprises:
a) a solid support comprising at least one capture reagent attached thereto, wherein the capture reagent binds to at least one biomarker selected from the group of polypeptides as above specified, and
b) instructions for using the said solid support to detect one or more of the at least one biomarker.

In a third embodiment, the kit comprises a solid support comprising one or more capture reagents attached thereto, wherein each of the capture reagent(s) is able to form a complex with a biomarker selected from the group of polypeptides as above specified.

According to the said third embodiment, the kit further comprises means for detecting the formation of complexes between a capture reagent attached to the said solid support and one of the said biomarkers.

In some embodiments, the said capture reagents consist of ligand molecules that specifically bind to one of the said biomarkers.

### BRIEF DESCRIPTION OF THE FIGURES

FIG. 1: Mean peak intensities of selected peaks (CM10-7558, CM10-7924 and Q10-7926) according to severity of liver lesions and after bariatric surgery in obese patients.

FIG. 2: Mean peak intensity of selected peaks (CM10-7558, CM10-7924 and Q10-7926) according to steatosis amount in obese patients.

FIG. 3: Mean peak intensity of selected peaks (CM10-7558, CM10-7924 and Q10-7926) according to NASH score in obese patients.

FIG. 4: Correlation between CM10-7558.4 and CM10-7924-2 peak intensity in obese patients.

FIG. 5A: Serum protein profiles of obese patients with non significant lesion (upper) and obese with NASH (lower) in CM10 ProteinChip

In serum of obese patient with NASH (lower panel), hemoglobin-α subunit appears as a double charged ion at m/z=7558 Da (left double arrow) and 15.126 Da (left simple arrow); hemoglobin-β subunit double charged ion is detected as a peak at 7924 Da (right double arrow) and 15.857 Da (right simple arrow). These peaks are not detected in serum of obese patient without liver lesion (upper panel).

FIG. 5B: Serum protein profiles of obese patients with NASH (upper) and obese with non significant lesion (lower) in Q10 ProteinChip. 7926 Da (arrow) is detected in upper profile.

FIG. 6: Characterization of protein peaks using immuno-SELDI in a serum of obese patient with NASH

Upper panel: Protein-Chip precoupled with antiserum against hemoglobin-α selectively retented a protein, that, after desorption and ionization-TOF produced a spectrum with 2 peaks at m/z=7558 Da (left arrow) and 15.126 Da (right arrow). These peaks were not observed in presence of PBS. Chip coated with non-immune IgG in presence of same serum did not captured any of these peaks

Lower panel: same experiment with proteinChip coupled with a specific antibody against hemoglobin-β. Two peaks at 7924 Da (left arrow) and 15.857 Da (right arrow).

### DETAILED DESCRIPTION OF THE INVENTION

It has been found, according to the invention, some new polypeptide biomarkers which are usable in methods or kits to determine the occurrence of a liver disease, in particular hepatitis and more precisely of Non-alcoholic steatohepatitis (NASH) type, in a subject.

### 1.—Introduction

A biomarker is an organic biomolecule, the presence of which in a sample is used to determine the occurrence of a disease and in particular the phenotypic status of the subject (e.g., patient affected of a liver disease v. normal or non-affected patient).

In a preferred embodiment, the biomarker is differentially present in a sample taken from a subject of one phenotypic status (e.g., having a disease) as compared with another phenotypic status (e.g., not having the disease).

In another preferred embodiment, the biomarker is also quantitatively differentially present in a sample taken from a subject to another, to determine the severity of the disease (e.g., from steatosis, steatohepatitis, fibrosis to cirrhosis).

A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio.

Biomarkers, alone or in combination, provide measures of relative risk that a subject belongs to one phenotypic status or another. Therefore, they are useful as markers in particular for disease (diagnostics), therapeutic effectiveness of a drug (theranostics), drug toxicity and evaluate chirurgical efficiency (e.g., bariatric surgery).

### 2.—Biomarkers According to the Invention

The invention provides, among other useful features, polypeptide-based biomarkers that are useful for determining the occurrence of a liver disease, in particular of the hepatitis type, in a subject.

These polypeptide biomarkers are differentially present in subjects having the liver disease versus healthy individuals.

These polypeptide biomarkers are also quantitatively present in a biological sample from the subject, e.g. in a serum sample, depending on the severity of the liver disease. In particular, these biomarkers can be used to diagnose between steatosis, steatohepatitis (in particular non-alcoholic steatohepatitis), fibrosis or cirrhosis.

Clinical proteomic is a validated approach that has enabled the determination of new serum biomarkers in several diseases.

The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a specific detected biomolecule is a biomarker of this invention. These characteristics represent inherent characteristics of the biomolecules and not process limitations in the manner in which the biomolecules are discriminated.

The biomarkers were identified herein using SELDI technology employing ProteinChip arrays from Ciphergen Biosystems, Inc. (Fremont, Calif.).

Serum samples were collected from candidates for bariatric surgery, before and after said surgery, and from non-obese individuals.

The samples were thawed and diluted in denaturing buffer. Serum samples were then processed using SELDI biochips and spectra of polypeptides in the samples were generated by time-of-flight mass spectrometry on a Ciphergen ProteinChip Reader.

The spectra thus obtained were analyzed by Ciphergen ProteinChip software version 3.0.2. For each comparison, raw intensity data were normalized using the total ion current of all profiles and logarithm transformed. To characterize protein peaks of potential interest differentially expressed in various phenotype groups of patients, the logarithm-transformed intensity of each protein peak was compared according to groups using non-parametric tests. Peaks were considered as significantly different for a p-value less than 0.05. Correlations between quantitative data were studied using coefficient of correlation.

This method is described in more detail in the Example Section.

The polypeptide-based biomarkers thus discovered are the following ones:
(i) a positively charged polypeptide at pH 5 having a SELDI-TOF mass-to-charge (m/z) ratio of 7558.4+/−0.3%, which positively charged polypeptide may also be termed CM10-7558,
(ii) a positively charged polypeptide at pH 5 having a SELDI-TOF mass-to-charge (m/z) ratio of 7924.2+/−0.3%, which positively charged polypeptide may also be termed CM10-7924, and
(iii) a negatively charged polypeptide at pH 6 having a SELDI-TOF mass-to-charge (m/z) ratio of 7926.9+/−0.3%, which negatively charged polypeptide may also be termed Q10-7926.

The biomarkers of this invention are in particular characterized by their mass-to-charge ratio as determined by mass spectrometry.

The mass-to-charge ratios were determined from mass spectra generated on a Ciphergen ProteinChip Reader (series 4000, Ciphergen Biosystems). This instrument has a mass accuracy of about +/−0.3 percent. Additionally, the instrument has a mass resolution of about 700 m/dm, where m is mass and dm is the mass spectral peak width at 0.5 peak height. The mass-to-charge ratio of the biomarkers was determined using externally calibrated standards (All-in-one Protein II) and ProteinChip software version 3.0.2 (Ciphergen Biosystems, Inc). Accordingly, the masses provided reflect these specifications.

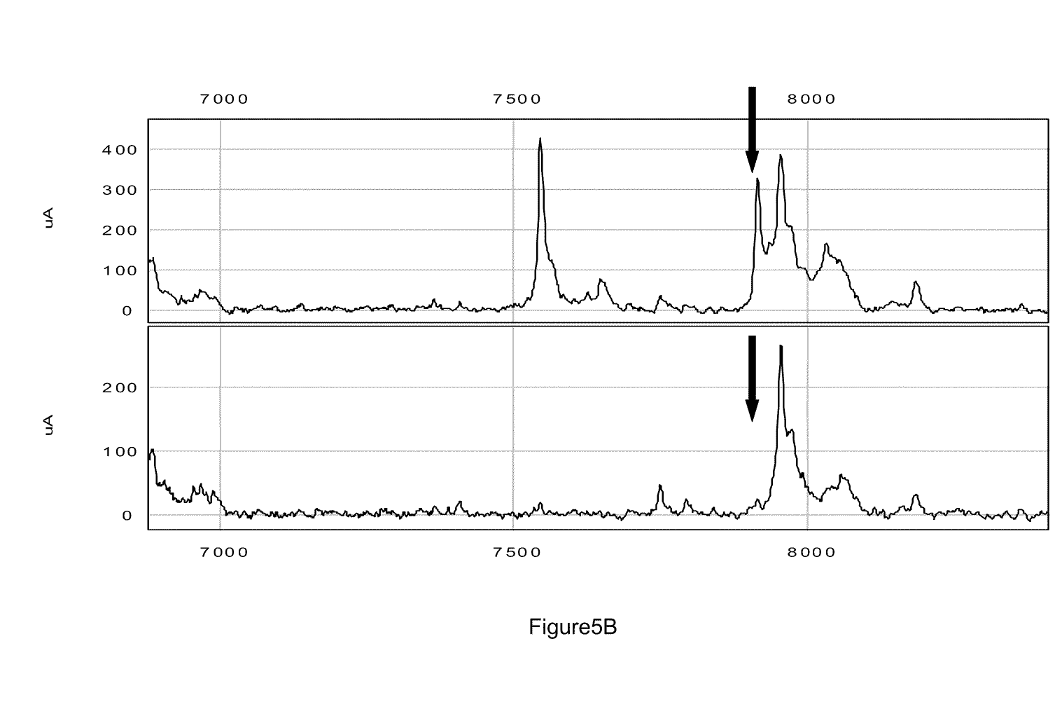

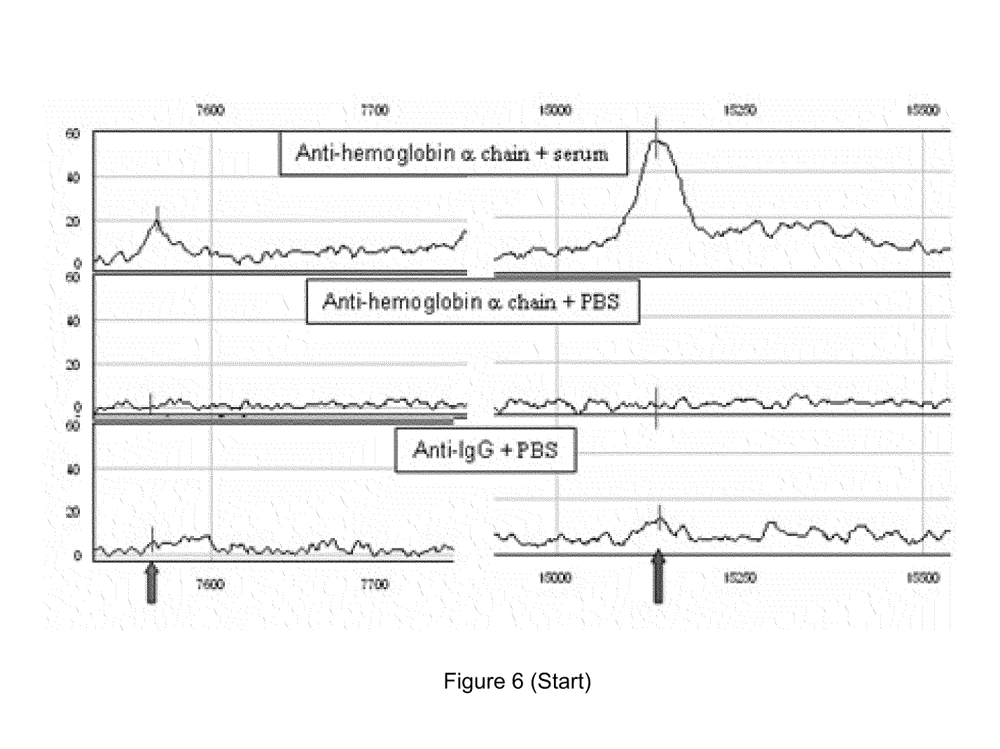

The biomarkers of this invention are further characterized by the shape of their spectral peak in time-of-flight mass spectrometry. Mass spectra showing peaks representing the biomarkers are presented in FIGS. 5A and 5B.

The biomarkers of this invention are further characterized by their binding properties on chromatographic surfaces.

In particular:
the biomarker CM10-7558 is a polypeptide which is able to bind to cationic exchange adsorbents (e.g., the Ciphergen® CM10 ProteinChip® array) after washing as follows: 2×200 μl washes with NaAc 50 mM pH 5, triton 0.1%; 1×200 μl NaAc 50 mM pH 5 and 2 rinses with 200 μl HEPES 1 mM,
the biomarker CM10-7924 is a polypeptide, which is also able to bind to cationic exchange adsorbents (e.g., the Ciphergen® CM10 ProteinChip® array) after washing with 2×200 μl washes with NaAc 50 mM pH 5, triton 0.1%; 1×200 μl NaAc 50 mM pH 5 and 2 rinses with 200 μl HEPES 1 mM,
the biomarker Q10-7926 is a polypeptide, which is able to bind to anionic exchange adsorbents (e.g., the Ciphergen® Q10 ProteinChip® array) after washing with 2×200 μl NaAc 50 mM pH 6, triton 0.1%; 1×200 μl NaAc 50 mM pH 6 and 2 rinses with 200 μl HEPES 1 mM.

Because the biomarkers of this invention are characterized by mass-to-charge ratio, binding properties and spectral shape, they can be detected by mass spectrometry without knowing their specific identity.

However, without excluding any further results of identification, the very close m/z values between CM10-7924 and Q10-7926 suggested that they are both related to the same protein that binds to Protein arrays with different affinities. Furthermore, it was observed a significant and strong correlation between peak intensity of CM10-7924.2 and CM10-7558 (r=0.84, p<0.001). This also suggested that these 2 peaks were different forms related to the same protein species. According to m/z ratio, it was assumed that CM10-7558 and CM-7924 were the double charged ions of hemoglobin-α (calculated mass: 15.126 kDa) and hemoglobin-β (calculated mass: 15.857 kDa) subunits, respectively. To validate this assumption, it was performed an immuno-SELDI assay, using antiserum against hemoglobin-α and with a specific antibody against hemoglobin-β. These results strongly suggest that CM10-7924 and CM10-7558 correspond to the double charged ions of α and β-hemoglobin subunits.

If desired, the biomarkers identity can be also characterized by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes.

Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the biomarkers is serum.

The biomarkers of this invention are biomolecules, and are more precisely polypeptides. Accordingly, this invention provides these biomolecules in isolated form. The biomarkers can be isolated from biological fluids, such as serum. They can be isolated by any method known in the art, based on both their mass and their binding characteristics.

For example, a sample comprising the biomolecules can be subject to chromatographic fractionation, as described herein, and subject to further separation by, e.g., acrylamide gel electrophoresis.

Knowledge of the identity of the biomarker also allows their isolation by immunoaffinity separation techniques, as it is shown in the examples herein.

### 3.—Detection of Biomarkers for Determining the Occurrence of a Liver Disease in a Subject

The biomarkers of this invention can be detected by any suitable method.

Detection methods that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

### 3.1.—Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

### 3.1.1.—SELDI

A preferred mass spectrometric technique for use in the invention is “Surface Enhanced Laser Desorption and Ionization” or “SELDI,” as described, for example, in U.S. Pat. No. 5,719,060 and U.S. Pat. No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with carboimidizole, expoxide) and PG-20 (protein G coupled through carboimidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); PCT International Publication No. WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (HCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of [alpha]-cyano-4-methacryloyloxycinnamic acid and acrylate. In another embodiment, the composition is a co-polymer of [alpha]-cyano-4-methacryloyloxycinnamic acid, acrylate and 3-(tri-ethoxy)silyl propyl methacrylate. In another embodiment, the composition is a co-polymer of [alpha]-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

### 3.1.2.—Other Mass Spectrometry Methods

In another mass spectrometry method, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

### 3.1.3.—Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline substraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### 3.1.4.—General Protocol for SELDI Detection of Biomarkers for Determining the Occurrence of Liver Disease

A preferred protocol for the detection of the biomarkers of this invention is as follows.

The biological sample to be preferably tested, e.g., serum, is subject to thawing and dilution in denaturing buffer before SELDI analysis.

The sample to be tested is then contacted with an affinity capture probe comprising a cation exchange adsorbent (preferably CM10, ProteinChip array (Ciphergen Biosystems, Inc.) or anion exchange adsorbent (preferably Q10, ProteinChip array (Ciphergen Biosystems, Inc.)). The CM10 array spots were preactivated with HCl 10 mM.

The probe is washed with a buffer that will retain the biomarker while washing away unbound molecules.

A suitable wash for each biomarker is the buffer identified (NaAc 50 mM ph 5, triton 0.1%).

The biomarkers are detected by laser desorption/ionization mass spectrometry.

Alternatively, if antibodies that recognize the biomarker are available, for example antibodies against α-hemoglobin subunit (e.g. antiserum H80, rabbit polyclonal IgG against α1-hemoglobin subunit, Santa Cruz Biotechnology, Santa Cruz, Calif.) or against β-hemoglobin subunit (e.g. antiserum H76, rabbit polyclonal IgG against β-hemoglobin subunit, Santa Cruz Biotechnology, Santa Cruz, Calif.), these can be attached to the surface of a probe, such as epoxy-activated PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

### 3.2.—Detection by Immunoassay

In another embodiment, the biomarkers of this invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

For example, it could be used antibodies against α-hemoglobin subunit (e.g. antiserum H80, rabbit polyclonal IgG against α1-hemoglobin subunit, Santa Cruz Biotechnology, Santa Cruz, Calif.) or against β-hemoglobin subunit (e.g. antiserum H76, rabbit polyclonal IgG against β-hemoglobin subunit, Santa Cruz Biotechnology, Santa Cruz, Calif.).

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### 4.—Determination of the Occurrence of a Liver Disease in a Subject

### 4.1.—Single Markers

The biomarkers of the invention can be used in diagnostic tests to determine the occurrence of liver disease in a subject, e.g., to diagnose liver disease status.

The words “liver disease” relate in particular to hepatitis, and include distinguishing, inter alia, subject having liver disease v. subject not having such a liver disease. They also relate to group consisting of a chronic liver disease and a non-chronic liver disease, or to group consisting of (i) a subject infected by a hepatitis virus, (ii) an alcoholic subject and (iii) a subject affected with obesity.

The diagnostic test includes also determining the severity of the liver disease, and in particular distinguishing between steatosis, steatohepatitis (in particular non-alcoholic steatohepatitis or NASH), and potentially also fibrosis and cirrhosis

Based on this status, further procedures may be indicated, including additional diagnostic tests or therapeutic procedures or regimens.

The power of a diagnostic test to correctly predict the occurrence disease is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

To validate these biomarkers, it was assessed the classification of future samples with different models and the leave-one-out cross validation procedure. For prediction of NASH according to serum protein profile, 75% of patients were correctly classified for the presence of NASH. Sensitivity, specificity, positive predictive value and negative predictive value were 0.83, 0.67, 0.71 and 0.8, respectively. For prediction of steatosis, the percentage of correctly classified was also 75%. Sensitivity was 0.78 and specificity was 0.71 (values were obtained for the marker CM 7598-4).

Each of the biomarkers described herein is individually useful for determining the occurrence of a liver disease.

The method involves, first, providing a sample previously collected from the subject, second, measuring at least one the above-mentioned biomarkers in the said sample using at least one of the methods described herein, third, determining the occurrence of a liver disease from the biomarker values measured in second step.

The values measured represent a measured amount of a biomarker allowing to determine a particular liver disease status of the tested subject.

In a preferred embodiment of the present invention, the occurrence and the severity of the liver disease is advantageously determined by measuring the amount of at least one of the biomarkers described herein.

Specifically, the increase of the severity of the liver disease is correlated to the increase of the amount of biomarkers in accordance to the invention.

The amount of the at least one biomarker is advantageously determined in accordance of at least one of the methods described herein, e.g. by determining the intensity value of the signal that is generated by the said biomarker when the said sample is subjected to a mass spectrum analysis or to a SELDI-TOF analysis.

The measured amount is then submitted to a classification algorithm or compared to a reference amount and/or pattern of biomarkers that is associated with the particular stage of the disease.

Also in a preferred embodiment, the severity of the liver disease is determined by performing the following steps: first, determining the intensity value of the signal that is generated by the said biomarker when the said sample is subjected to a SELDI-TOF analysis, second, comparing the signal intensity value obtained at first step with at least a reference signal intensity value that is expected to be measured in an individual selected form the group consisting of (i) an individual who is not affected by a liver disease, (ii) an individual who is affected with a liver steatosis, (iii) an individual who is affected with a steatohepatitis, and (iv) an individual who is affected with a cirrhosis, and third, determining the severity of the liver disease of the tested subject.

In this last embodiment, the severity of the liver disease may be determined in a third step as being the status for which the ratio of the subject to the reference signal intensity values are close to 1, the said ratio preferably ranging from 0.99 to 1.01.

### 4.2.—Combination of Markers

While individual biomarkers are useful diagnostic biomarkers, a combination of markers can also provide greater predictive value of a particular status.

Specifically, the detection of a plurality of biomarkers in a sample can increase the sensitivity and/or specificity of the test.

### 4.3.—Subject Management

In certain embodiments of the methods of determining the occurrence of liver disease, the methods further comprise managing subject treatment based on the status. Such management includes the actions of the physician or clinician subsequent to determining liver disease status.

For example, if a physician makes a diagnosis of liver disease in an obese subject who is candidate to bariatric surgery, the biomarkers allow to monitoring the post-operative status of the patient, to following liver changes after weight loss or corrections of metabolic troubles.

### 5.—Kits for Detection of Biomarkers for Determining the Occurrence of Liver Disease

In another aspect, the present invention provides kits for qualifying liver disease status, which kits are used to detect and/or quantify biomarkers according to the invention.

In one embodiment, the kit comprises a solid support, such as a chip, a microtiter plate or a bead or resin having a capture reagent attached thereon, wherein the capture reagent binds a biomarker of the invention.

Thus, for example, the kits of the present invention can comprise mass spectrometry probes for SELDI such as ProteinChip® arrays. In the case of biospecific capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

The kit may also comprise means for detecting the formation of complexes between a capture reagent attached to the said solid support and one of the said biomarkers.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### 6. Use of Biomarkers for Determining the Occurrence of Liver Disease in Screening Assays

The methods of the present invention have other applications as well. For example, the biomarkers can be used to screen for compounds that decrease the expression of the biomarkers in vitro or in vivo, which compounds in turn may be useful in treating or preventing liver disease, in particular of hepatitis, in patients.

In another example, the biomarkers can be used to monitor the response to treatments for liver disease.

Thus, for example, the kits of this invention could include a solid substrate having a cationic or anionic function, such as a protein biochip (e.g., a Ciphergen CM10 or Q10 ProteinChip array, e.g., ProteinChip array) and a buffer for washing the substrate, as well as instructions providing a protocol to measure the biomarkers of this invention on the chip and to use these measurements to diagnose liver disease status.

In an aspect, the invention provides a method for identifying compounds useful for the treatment of disorders such as liver disease which are associated with increased levels of the biomarkers.

In yet another embodiment, the invention provides a method for treating or reducing the progression or likelihood of the liver disease.

At the clinical level, screening a test compound includes obtaining samples from test subjects before and after the subjects have been exposed to a test compound. The levels in the samples of one or more of the biomarkers may be measured and analyzed to determine whether the levels of the biomarkers change after exposure to a test compound. The samples may be analyzed by mass spectrometry, as described herein, or the samples may be analyzed by any appropriate means known to one of skill in the art.

For example, the levels of one or more of the biomarkers may be measured directly by Western blot using radio- or fluorescently-labeled antibodies which specifically bind to the biomarkers.

Subjects who have been treated with test compounds will be routinely examined for any physiological effects which may result from the treatment. In particular, the test compounds will be evaluated for their ability to decrease disease likelihood in a subject.

Alternatively, if the test compounds are administered to subjects who have previously been diagnosed with liver disease, test compounds will be screened for their ability to slow or stop the progression of the disease.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### EXAMPLES

### A. Materials and Methods

### A.1. Patients

Our series included 80 consecutive candidates for bariatric surgery who were prospectively included. Patients were selected on the basis of high BMI (>32 kg/m2), absence of significant alcohol consumption (<200 g per week for men and 100 g for women) and negative autoimmune and viral hepatitis work-ups. Written informed consent was obtained for all patients. A blood sample, collected before bariatric surgery, was performed to determine serum levels of alanine aminotransferase (ALT), aspartate aminotransferase (AST), γ-glutamyltransferase (GGT), alkaline phosphatase, glucose, total cholesterol and triglyceride. A serum aliquot was also immediately frozen for proteomic analysis and stored at −80° C. until use.

Bariatric surgery consisted of either gastric bypass or gastroplasty. Wedge liver biopsy was systematically performed during surgery. Thirty-three of 80 patients were reviewed 6 months after surgery. They were weighed and a blood sample was collected for proteomic analysis.

A control group of 24 non-obese individuals was designated among blood donors fulfilling the inclusion criteria concerning alcohol consumption and viral hepatitis tests. For each of them, blood samples were collected and tested for proteomic analysis.

### A.2. Pathological Analysis

Liver biopsy was routinely processed for histological analysis. Slides were stained with hematoxylin-eosin, Picrosirius red and Masson's trichrome for fibrosis evaluation. Scoring systems were adapted from Brunt and Kleiner's criteria. Steatosis was scored as absent (<10%), mild (10-30%), moderate (30-60%) or severe (>60% of hepatocytes), respectively. Portal and lobular inflammatory infiltrates were graded as absent (0), mild (1) or significant (2). Fibrosis were staged according to Kleiner et al.: no fibrosis (0), zone 3 perisinusoidal or portal fibrosis (1), perisinusoidal and portal fibrosis without bridging (2), bridging fibrosis (3) and cirrhosis (4). The presence of apoptotic bodies, ballooning degeneration and Mallory's hyaline was noted (0=absence, 1=presence). The diagnosis of NASH was based on a combination of steatosis and either lobular inflammation with ballooning degeneration or extensive fibrosis (stage >2). A score of NASH was obtained by adding semi-quantitative evaluation of inflammation, liver cell ballooning, and fibrosis.

### A.3. Proteomic Analysis

A total of 137 serum samples, comprising 80 samples belonging to obese patients before bariatric surgery (S1), 33 samples collected 6 months after surgery (S2) and 24 samples belonging to the control group were analyzed.

Each serum aliquot was thawed and diluted (1:10) in denaturing buffer (urea 7M, thiourea 2M, CHAPS 4%, dithioerythriol 0.1%).

Serum samples were processed using 3 types of chip arrays (cationic exchange /CM10, anionic exchange /Q10 and immobilized metal ion affinity capture /IMAC30 ProteinChip array loaded with zinc) according to the manufacturer's protocols (Ciphergen ProteinChip Reader, Ciphergen, Biosystems, Inc. Freemont, Calif., USA).

The IMAC30 and CM10 array spots were preactivated with ZnCl 100 mM for 10 min and HCl 10 mM for 5 min, respectively.

Five μl of each diluted serum was spotted onto array chips and incubated with 95 μl of binding buffer (AcNa 50 mM ph 5, triton 0.1%; PBS 1×, NaCl 0.5M, triton 0.1%) for 30 min. After washing and air drying, arrays were saturated with sinapinic acid in 0.5% trifluoroacetic acid and 50% acetonitrile. All samples were tested during the same experiment.

The arrays were analyzed with the Ciphergen ProteinChip Reader (series 4000, Ciphergen Biosystems). The mass spectra of proteins were generated using an average of 265 laser shots. Focus mass and laser intensity were adapted for low (3-10 kDa), intermediate (10-20 kDa) and high (20-200 kDa) molecular weight proteins. The mass-to-charge ratio (m/z) of proteins captured on the array surface was determined according to externally calibrated standards (All-in-one Protein II).

Intra-ProteinChip array reproducibility was checked by spotting 8 different aliquots of one sample on the same array, and inter-ProteinChip array reproducibility was checked by including one sample on each array. The mean of the 3 inter-ProteinChip arrays coefficient of variations ranged from 29.2 to 37.7% and 29.3 to 33% for low and intermediate molecular weight proteins, respectively.

### A.4. Statistical Analysis

Spectra were analyzed with ProteinChip software version 3.0.2 (Ciphergen Biosystems). For each comparison, raw intensity data were normalized using the total ion current of all profiles and logarithm transformed. To characterize protein peaks of potential interest differentially expressed in various phenotype groups of patients, the logarithm-transformed intensity of each protein peak was compared according to groups using non-parametric tests. Peaks were considered as significantly different for a p-value less than 0.05. Correlations between quantitative data were studied using coefficient of correlation.

To validate the results, the class of future samples was predicted according to their protein profiles using several models: compound covariate predictor, diagonal linear discriminant analysis, nearest neighbor classification, and support vector machines with linear kernel. The prediction error of each model was estimated using the leave-one-out cross-validation (LOOCV). For each LOOCV training set, the entire model building process was repeated, including the peak selection process. It was also evaluated whether the cross-validated error rate estimate for a model was significantly less than one would expect from random prediction. The class labels were randomly permuted and the entire LOOCV process was repeated.

### A.5. Identification of Biomarkers

To confirm the identity of potential biomarkers, epoxy-activated protein chips (PS20, Ciphergen) were precoupled with antiserum H80 (rabbit polyclonal IgG against α1-hemoglobin subunit, Santa Cruz Biotechnology, Santa Cruz, Calif.), or antiserum H76 (rabbit polyclonal IgG against β-hemoglobin subunit, Santa Cruz Biotechnology, Santa Cruz, Calif.) or non immune rabbit IgG (Santa Cruz Biotechnology, Santa Cruz, Calif.). Antibody (4 μl, 0.2 mg/ml) was applied to protein chip and mixed with 2 μl 50 mM NaHCO3 buffer (pH 9.2). After shaking for 2 h at room temperature in a humid chamber, spots were washed (0.5 M ethanolamine (pH 8) for 1 h, and then PBS containing 0.5% triton X100). Serum samples, diluted 2-fold in PBS containing 0.1% triton X-100, were applied to the spot. The chips were incubated for 2 h, and wells were washed 2 folds with PBS containing 0.1% triton X-100, then with 50 mM Tris HCl, 1M urea, 0.1% 3-[(3-cholamidopropyl) dimethylammonio]-1 propanesulfate and 0.5 M NaCl (pH 7.2), and then with HEPES 5 mM (pH 7.2) before air drying. Sinapinic Acid (20 mg/ml in 50% acetonitrile and 0.5% TFA, 2×0.5 μl) was added to each spot and dried. Protein mass spectra were generated.

### Example

### Identification of the Polypeptide Biomarkers of the Invention

### Clinical and Biological Data

The group of obese patients comprised 60 women and 20 men with a mean age of 37 years (19-67 years). The mean BMI upon admission was 42 kg/m2 (37-47). The mean BMI measured 6 months after bariatric surgery was 30 kg/m2 (26-34). The mean BMI of the control group was 24 kg/m2 (21.5-26.5). Detailed biological data are presented in table 1.

TABLE 1
Patients clinical and biological data

| | Obese patients | Obese patients | |
|---|---|---|---|
| | before bariatric | after bariatric | Non-obese |
| | surgery | surgery | controls |
| | (n = 80) | (n = 33) | (n = 24) |
| Age | 35 ± 12 | | 30 ± 10 |
| Female/male | 60/20 | 25/8 | 11/19 |
| ratio | | | |
| BMI (kg/m²) | 42 ± 5.5 | 30 ± 4 | 24 ± 2.5 |
| Diabetes | 21 | | 0 |
| Fasting glucose | 6.4 ± 3 | 5.2 ± 0.7 | NA |
| (mmol/l) | | | |
| Cholesterol | 5.5 ± 1 | 5.0 ± 1.3 | NA |
| (mmol/l) | | | |
| Triglycerides | 1.9 ± 0.9 | 1.3 ± 0.6 | NA |
| (mmol/l) | | | |
| ASAT (IU/l) | 33.7 ± 18.3 | 20.6 ± 5.6 | NA |
| ALAT (IU/l) | 44.7 ± 33.2 | 26.8 ± 11.2 | NA |
| Alkaline | 72.2 ± 13.8 | 68.7 ± 15.9 | NA |
| phosphatase | | | |
| (IU/l) | | | |

NA: not available

### Liver Biopsy Analysis

Eighty biopsy specimens belonging to obese patients were collected at the time of bariatric surgery. Steatosis was present (>10%) in 56 patients (70%), and 23 (29%) had severe steatosis (>60%). When steatosis was mild or moderate, it was predominantly located in the centrolobular area. Portal and lobular inflammation was present to a mild degree in 44 (55%) and 39 cases (49%), respectively. Ballooning degeneration was found in 6 cases (7.5%) and apoptotic bodies were detected in 25 cases (31%). Fibrosis was staged 0 in 33 cases (41%), 1 in 24 cases (30%), 2 in 17 cases (21%) and 3 in 6 cases (8%). There were no cases of cirrhosis.

Finally, 24 patients (30%) were considered as having non significant liver lesion, 32 (40%) as significant steatosis alone (without NASH) and 24 (30%) as NASH.

Main histopathologic data are shown in table 2.

TABLE 2
Main histological findings in obese patients before bariatric surgery

| | | Number of biopsy |
|---|---|---|
| Histological features | Category | specimens (%) |
| Steatosis | No (<10%) | 24 (30%) |
| | Mild (<30%) | 11 (14%) |
| | Moderate (30%-60%) | 18 (22%) |
| | Severe (>60%) | 23 (29%) |
| Fibrosis stage | 0 = None | 33 (41%) |
| | 1 = Sinusoidal or portal | 24 (30%) |
| | fibrosis | |
| | Sinusoidal (1A/1B) | 5/0 |
| | Portal (1C) | 19 |
| | 2 = Portal and sinusoidal | 17 (21%) |
| | 3 = Bridging fibrosis | 6 (8%) |
| | 4 = Cirrhosis | 0 (0%) |
| Diagnosis | Non significant | 24 (30%) |
| | Steatosis without NASH | 32 (40%) |
| | NASH | 24 (30%) |

### Proteomic Analysis

### 1. Comparison of Protein Profiles of Obese Patients and Controls

For each serum, a mean of 666 protein peaks were generated with the 3 Chip-arrays (IMAC 30, Q10 and CM10). Comparison of protein profiles obtained in the obese group before surgery (n=80) with those of the control group (n=24) showed a total of 112 differentially expressed protein peaks (p<0.01). Seventy-one were up-regulated in the obese group whereas 41 were down-regulated. Comparing the proteome profiles of the control group and those of the obese without significant liver lesions (n=24), 97 peaks significantly differed (p<0.05).

### 2. Comparison of Protein Profiles in Obese Patients According to Histopathological Lesions

In the group of obese patients, comparison of those having no significant liver lesions (n=24) with those with steatosis alone (n=32) identified 17 differentially expressed protein peaks (CM10/8, Q10/4 and IMAC30/5, p<0.05).

Eight proteins were upregulated and 9 were downregulated in the presence of significant steatosis. In a similar manner, comparison of serum protein profiles of obese patients with NASH (n=24) to those without significant liver lesions (n=24) showed 50 differentially expressed protein peaks (Q10/12, CM10/26 and IMAC30/12).

Forty-six proteins were upregulated and 4 were downregulated in NASH. Some of the peaks had very high p-values in different statistical tests (e.g. p<10-5) indicating strong statistical significance of differential expression.

Comparison of protein profile of the group of patients with steatosis without NASH to those with NASH identified 67 peaks differentially expressed (CM10/22, IMAC/24 and Q10/21). Fifty were upregulated and 17 downregulated when serum proteome profiles of patients with NASH were compared to those with steatosis.

To validate the results, we assessed the classification of future samples with different models and the leave-one-out cross validation procedure. For prediction of NASH according to serum protein profile, 75% of patients were correctly classified for the presence of NASH. Sensitivity, specificity, positive predictive value and negative predictive value were 0.83, 0.67, 0.71 and 0.8, respectively. For prediction of steatosis, the percentage of correctly classified was also 75%. Sensitivity was 0.78 and specificity was 0.71.

3. Modifications in Protein Profiles after Weight Loss in Obese Patients

Comparison of serum protein profiles before and after bariatric surgery showed 94 significantly modified peaks. Sixty-two peaks were downregulated in the serum and 32 peaks were upregulated after weight loss. Six out of the 17 protein peaks which had been previously correlated with the presence of significant steatosis, as well as 17 of the 50 peaks associated with the diagnosis of NASH, returned to near normal values after bariatric surgery.

### 4. Serum Biomarkers of Liver Lesions in Obese Patients

Finally, 3 protein peaks (CM10-7558.4, CM10-7924.2 and Q10-7926.9) were found to be common when comparing peaks differentially expressed between obese patients without lesions to those with pure steatosis and those with NASH.

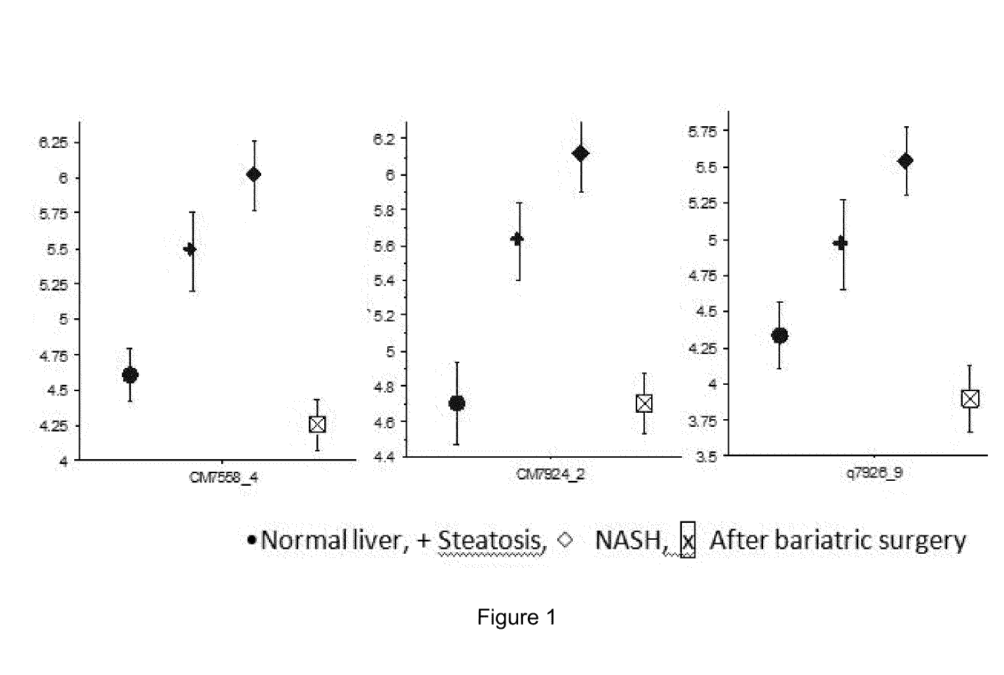

Interestingly, mean peak intensity progressively and significantly increased from no lesion to steatosis and then to NASH (p<0.001). Six months after bariatric surgery, all 3 peaks returned to values close to those of patients without liver lesions (FIG. 1).

Mean intensity of each of these 3 peaks was not significantly different when comparing the control group to obese patients without liver lesions. Furthermore, in the whole group of obese patients, no significant correlation was observed between any of the 3 peaks and laboratory parameters, either liver function tests or metabolic parameters.

Taken together, these results demonstrate that CM10-7558.4, CM10-7924.2 and Q10-7926.9 are serum biomarkers mirroring liver lesions rather than severity of serum metabolic disturbances or liver test abnormalities.

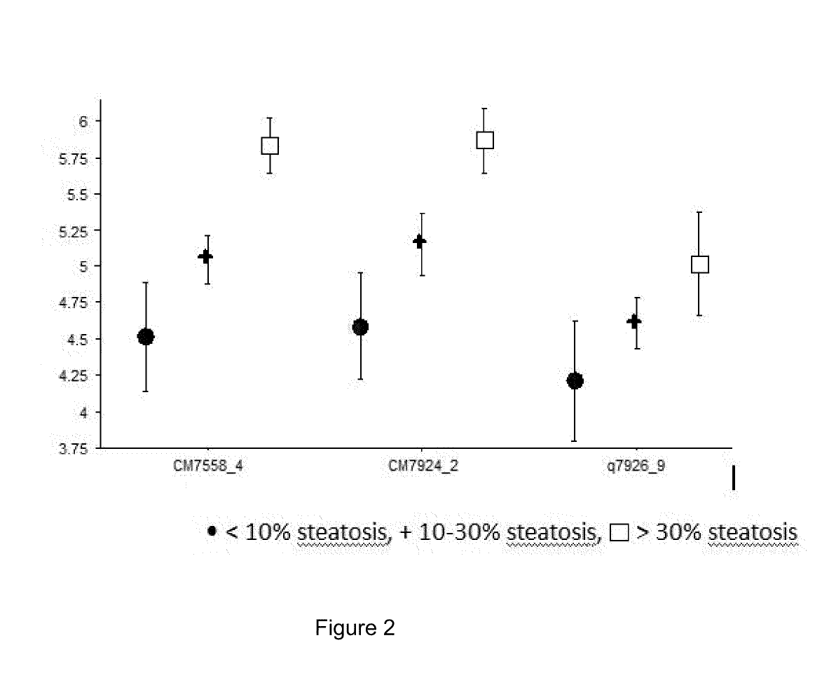

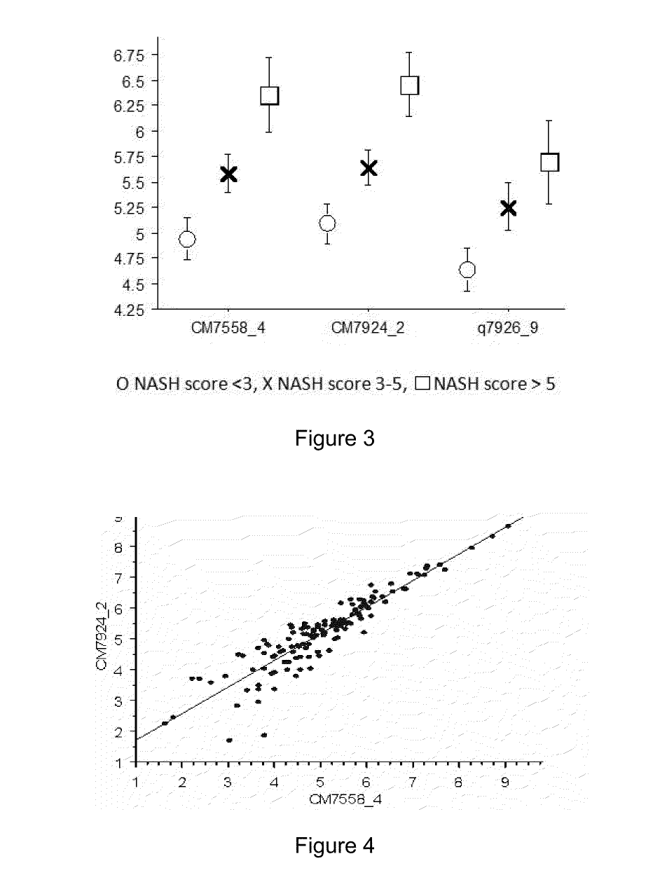

Finally, peak intensities of CM10 7558.4, CM10 7924.2 and Q10 7926.9 were then compared in the group of 80 obese patients according to steatosis intensity (<10%, n=24, 10-30%, n=11 and >30%, n=41). A clear significant increase of all peaks was observed according to steatosis amount (p<0.001 for each peak) (FIG. 2). Similarly, CM10 7558.4, CM10 7924.2 and Q10 7926.9 peak intensity also increased according to a NASH scoring system (p<0.01 for CM10 7558.4 and CM10 7924.2 and p<0.05 for q-7926.9) (FIG. 3).

### Identification of Biomarker

The very close m/z values between CM10-7924 and Q10-7926 suggested that they were both related to the same protein that binds to Protein arrays with different affinities.

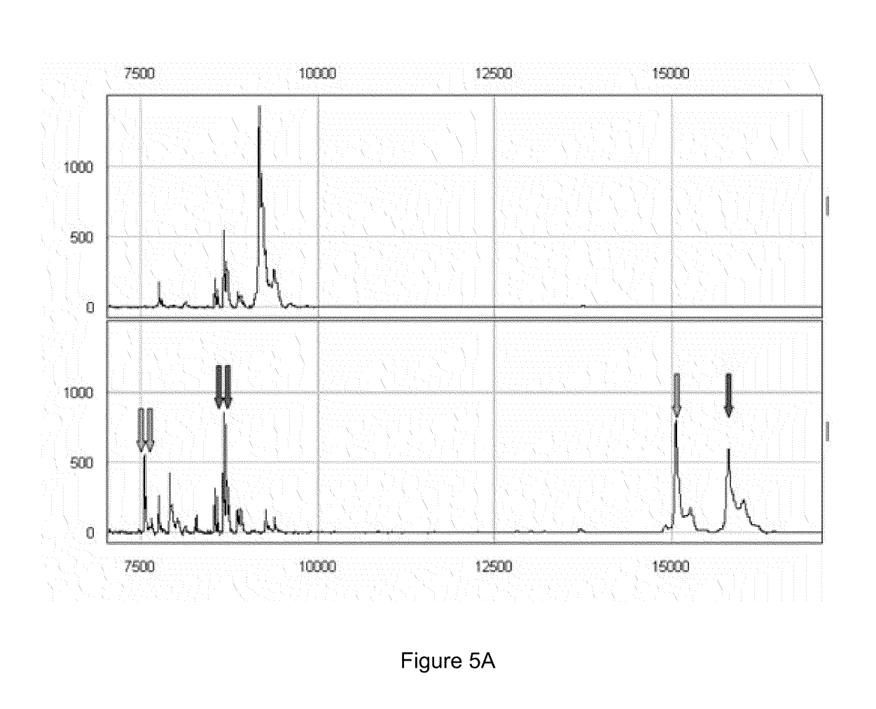

Furthermore, we observed a significant and strong correlation between peak intensity of CM-7924.2 and CM-7558 (r=0.84, p<0.001) (FIG. 4).

This also suggested that these 2 peaks were different forms related to the same protein specie.

According to m/z ratio, we assumed that CM-7558 and CM-7924.2 were the double charged ions of hemoglobin-α (calculated mass: 15.126 kDa) and hemoglobin-β (calculated mass: 15.857 kDa) subunits, respectively. These two peaks were also represented by 2 prominent peaks in the SELDI-spectra (FIG. 5).

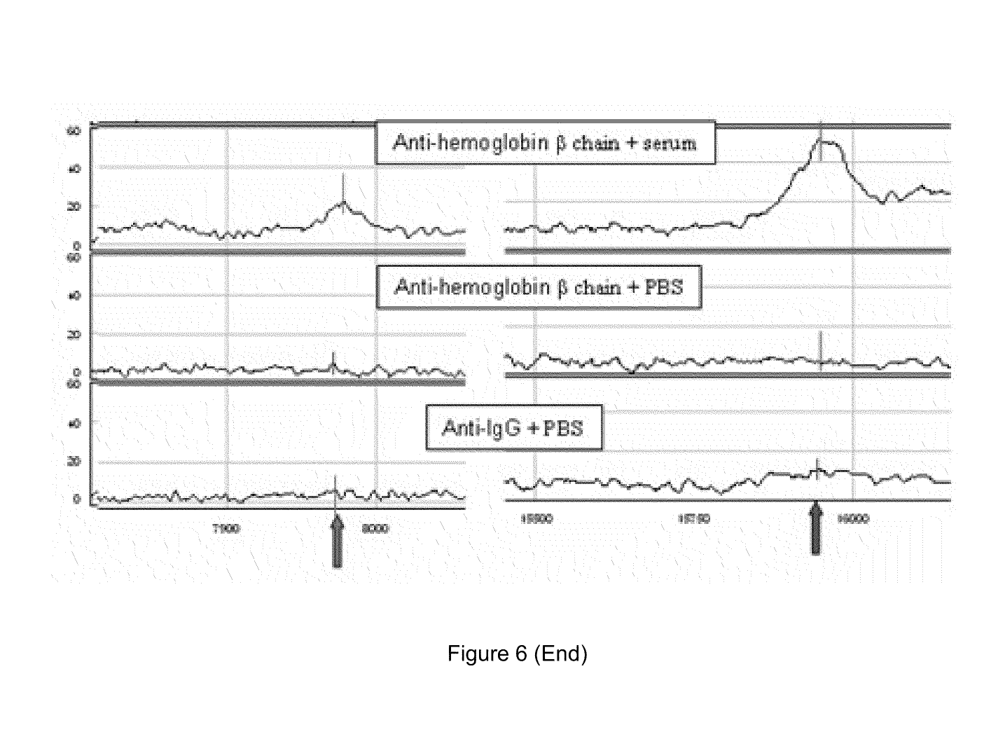

To validate this assumption, we performed an immuno-SELDI assay. As shown in FIG. 6, protein-Chip precoupled with antiserum against hemoglobin-α selectively retented a protein, that, after desorption and ionization-TOF, produced a spectrum with 2 peaks at 7558 and 15.126 Da. Similarly, proteinChip coupled with a specific antibody against hemoglobin-β also produced a spectrum with 2 peaks at 7924 and 15.857 Da. Chip coated with non-immune rabbit IgG in presence of same serum or coated with either antibody against hemoglobin-α or -β in presence of PBS did not capture any of these peaks (also as shown FIG. 6).

These results strongly suggest that CM10-7924.2 and CM10-7558 correspond to the double charged ions of α and β-hemoglobin subunits.

In the present study, it is demonstrated that a proteomic-based method allows to outline changes in serum protein profiles in obese patients according to severity of liver lesions and that these profiles were significantly modified after weight loss, suggesting an improvement in liver lesions. Identification of free hemoglobin subunits as potential markers of severity of liver damages may serve as a serum biomarker of liver lesions.

In general, the biomarkers of the invention have the real interest of being correlated in amount with the severity of the liver disease, which is particularly useful to determine the disease status of the patient.

1. A method for determining the occurrence of a liver disease in a subject comprising the steps of:
a) providing a sample previously collected from the said subject,
b) measuring at least one biomarker in the said sample, wherein the said biomarker is selected from the group of polypeptides consisting of
(i) a positively charged polypeptide at pH 5 having a SELDI-TOF mass-to-charge (m/z) ratio of 7558.4+/−0.3%,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%,
c) determining the occurrence of a liver disease from the biomarker values measured at step b).

2. The method according to claim 1, wherein the liver disease is selected from the group consisting of a chronic liver disease and a non-chronic liver disease.

3. The method according to claim 1, wherein the chronic liver disease is selected from steatosis, steatohepatitis, cirrhosis and fibrosis.

4. The method according to claim 3, wherein steatohepatitis consists of Non-alcoholic steatohepatitis (NASH).

5. The method according to claim 1, wherein the said subject is selected from the group consisting of (i) a subject infected by a hepatitis virus, (ii) an alcoholic subject and (i) a subject affected with obesity.

6. The method according to claim 1, wherein the said sample consists of a serum sample that has been collected from the said subject.

7. The method according to claim 1, further comprising a step d) of determining the severity of the liver disease, wherein the severity of the liver disease is determined by measuring the amount of the at least one biomarker measured at step b).

8. The method according to claim 7, wherein measuring the amount of the at least one biomarker is performed by determining the intensity value of the signal that is generated by the said biomarker when the said sample is subjected to a mass spectrum analysis.

9. The method according to claim 7, wherein measuring the amount of the at least one biomarker is performed by determining the intensity value of the signal that is generated by the said biomarker when the said sample is subjected to a SELDI-TOF analysis.

10. The method according to claim 1, further comprising a step d) of determining the severity of the liver disease by performing the following steps of:
d1) determining the intensity value of the signal that is generated by the said biomarker when the said sample is subjected to a SELDI-TOF analysis,
d2) comparing the signal intensity value obtained at step d1) with at least a reference signal intensity value that is expected to be measured in an individual selected form the group consisting of (i) an individual who is not affected by a liver disease, (ii) an individual who is affected with a liver steatosis and (iii) an individual who is affected with a steatohepatitis,
d3) determining the severity of the liver disease.

11. The method according to claim 10, wherein the severity of the liver disease is determined at step d3) as being the status for which the ratio of the subject to the reference signal intensity values are close to 1.

12. The method according to claim 7, wherein the amount of the at least one biomarker is measured by performing an immunoassay.

13. A method for monitoring the post-operative status of a patient that has been subjected to a bariatric surgery comprising a step of performing the method according to claim 1 with one or more samples that have been collected from the said patient at one or more instants following the surgical operation.

14. A method for the in vivo testing of a substance for treating a liver disease comprising the steps of:
a) administering the said substance to a patient in need of a medical treatment of a liver disease,
b) performing the method according to claim 1 on the said patient, and
c) determining the effect of the said substance on the liver disease of the said patient.

15. The method according to claim 14, further comprising carrying out the following steps, before the administering step:
a) providing a sample previously collected from the said subject,
b) measuring at least one biomarker in the said sample, wherein the said biomarker is selected from the group of polypeptides consisting of
(i) a positively charged polypeptide at pH 5 having a SELDI-TOF mass-to-charge (m/z) ratio of 7558.4+/−0.3%,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%,
c) determining the occurrence of a liver disease from the biomarker values measured at step b).

16. A kit for determining the occurrence of a liver disease in a subject comprising means necessary for measuring at least one biomarker in the said sample, wherein the said biomarker is selected from the group of polypeptides consisting of
(i) a positively charged polypeptide at pH 5 having a SELDI-TOF mass-to-charge (m/z) ratio of 7558.4+/−0.3%,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%.

17. A kit for determining the occurrence of a liver disease in a subject comprising:
a) a solid support comprising at least one capture reagent attached thereto, wherein the capture reagent binds to at least one biomarker selected from the group of polypeptides consisting of
(i) a positively charged polypeptide at pH 5 having a SELDI-TOF mass-to-charge (m/z) ratio of 7558.4+/−0.3%,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%, and
b) instructions for using the said solid support to detect one or more of the at least one biomarker.

18. A kit for determining the occurrence of a liver disease in a subject comprising a solid support comprising one or more capture reagents attached thereto, wherein each of the capture reagent(s) is able to form a complex with a biomarker selected from the group of polypeptides consisting of
(i) a positively charged polypeptide at pH 5 having a SELDI-TOF mass-to-charge (m/z) ratio of 7558.4+/−0.3%,
(ii) a positively charged polypeptide at pH 5 having a SELDI-TOF mass-to-charge (m/z) ratio of 7924.2+/−0.3%, and
(iii) a negatively charged polypeptide at pH 6 having a SELDI-TOF mass-to-charge (m/z) ratio of 7926.9+/−0.3%,

19. The kit according to claim 18, further comprising means for detecting the formation of complexes between a capture reagent attached to the said solid support and one of the said biomarkers.

20. The kit according to claim 18 wherein the said capture reagents consist of ligand molecules that specifically bind to one of the said biomarkers.
